(19) United States
(12) Patent Application Publication
(10) Pub. No.: US 2016/0007600 A1
(43) Pub. Date: Jan. 14, 2016

(54) SYNTHETIC LIPOCHITOOLIGOSACCHARIDE ANALOGS FOR ENHANCING PLANT PERFORMANCE

(71) Applicant: E.I. DU PONT DE NEMOURS AND COMPANY, Wilmington, DE (US)

(72) Inventors: Zheng-Zheng Huang, Hockessin, DE (US); Anilkumar Raghavanpillai, Wilmington, DE (US); Subramaniam Sabesan, Wilmington, DE (US); Christina Jacy Spado, Philadelphia, DE (US); Zuohong Yin, West Chester, PA (US)

(21) Appl. No.: 14/770,654

(22) PCT Filed: Mar. 6, 2014
(86) PCT No.: PCT/US2014/021428

§ 371 Date: Aug. 26, 2015

Related U.S. Application Data
(60) Provisional application No. 61/773,226, filed on Mar. 6, 2013.

(51) Int. Cl. A01N 43/16; C07H 15/04

(52) U.S. Cl. CPC A01N 43/16; C07H 15/04

(57) ABSTRACT

Compounds that are related to natural synthetic lipochitooligosaccharide analogs were chemically synthesized and found to be effective as plant performance enhancers. The compounds are amine-oligosaccharides with a substituent aryl-containing aliphatic carboxylic acid attached at the amine to the terminal amine-sugar unit. The aryl containing aliphatic carboxylic acid terminates with an aromatic ring structure.

### FIELD OF THE INVENTION

The invention relates to synthetic compounds for enhancing plant performance.

### BACKGROUND

Lipochitooligosaccharides are naturally made in rhizobial bacteria and function as nodulation factors which increase productivity of a variety of crops, including soybeans, peanuts, alfalfa, and dry beans. The nodulation factor lipochitooligosaccharides have a backbone of four or five β1,4-linked N-acylated glucosamine residues, a structure also found in chitin (poly-[1-4]-β-N-acetyl-D-glucosamine). This backbone is N-acylated and can carry diverse substitutions at both ends, depending on the rhizobial species in which it is made. In some rhizobia the N-acylation of the terminal unit is with fatty acids of general lipid metabolism such as vaccenic acid (C18:1Δ11Z) and in other rhizobia the N-acylation is with polyunsaturated fatty acids such as C20:3 and C18:2. The nodulation factor lipochitooligosaccharides made in any one species of bacteria are a mixture of compounds having different substitutions that are not possible to completely separate. Preparations of lipochitooligosaccharides produced through biological means have variable activity.

Some nodulation factor lipochitooligosaccharides have been chemically synthesized. There are various reported methods for making small samples of lipochitooligosaccharides, for example as described in Nicolaou et al., J. Am. Chem. Soc. 114: 8701-8702 (1992); Ikeshita et al., Carbohydrate Research C1-C6 (1995); and Wang et al., J. Chem. Soc. Perkin Trans. 1: 621-628 (1994). U.S. Pat. No. 8,049,002 discloses a process of synthesizing individual lipochitooligosaccharide analogs that is amenable to large scale production.

U.S. Pat. No. 8,198,420 discloses synthetic compounds which are active as nodulation factors and as plant growth stimulators, that have an aromatic ring attached to an oligosaccharide, and a carbon chain attached to the aromatic ring.

There remains a need for additional compounds related to natural lipochitooligosaccharides that are effective plant performance enhancers, that can be provided by chemical synthesis.

### SUMMARY OF THE INVENTION

One aspect of the present invention provides a compound having the structure (I)

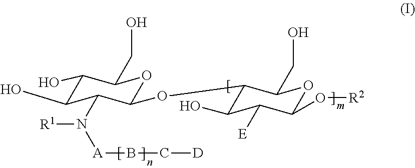

wherein the substituents are:

n is 1-20;

m is 0, 1, 2, 3 or 4;

A is selected from —C(O)—, —C(S)—, C(O)O—, —C(O)S—, —C(S)S—;

B is selected from —C≡C—, —CR³═CR⁴—, and combinations thereof, wherein when present the carbon-carbon double bond configuration is selected from cis, trans, and a mixture of cis and trans;

C is a substituted or unsubstituted arylene wherein there are one, two, three, or four substituents that are independently selected from a halogen, —CN, —C(O)OR⁵, —C(O)NR⁶R⁷, —CF₃, —OCF₃, —NO₂, —N₃, —OR⁵, —SR⁵, —NHR⁶, —NR⁶R⁷, and —C1-6 alkyl;

D is selected from H, CH₃, and a linear or branched, saturated or unsaturated, hydrocarbon-based chain containing from 2 to 20 carbon atoms;

E is selected from OH, NH₂, and NHC(O)CH₃;

R¹ is selected from H and C1-20 alkyl;

R² is selected from a linear or branched, saturated or unsaturated, hydrocarbon-based chain containing from 1 to 20 carbon atoms, arylene, or substituted arylene;

R³ and R⁴ are independently selected from C1-6 alkyl, a halogen, and an aryl; and

R⁵, R⁶ and R⁷ are independently selected from H, —C1-6 alkyl, —C(O)C1-6 alkyl, —C(S)C1-6 alkyl, —C(O)OC1-6 alkyl, —C(O)NH₂, —C(S)NH₂, —C(NH)NH₂, —C(O)NHC1-6 alkyl, —C(S)NHC1-6 alkyl, and —C(NH)NHC1-6 alkyl.

Another aspect of the present invention provides an agricultural composition comprising a compound of the structure (I).

Another aspect of the present invention provides a method of treating plant material comprising:

a) providing an agricultural composition comprising a compound of the structure (I); and

b) contacting plant material with the composition of (a).

### DETAILED DESCRIPTION

The following definitions and abbreviations are to be used for the interpretation of the claims and the specification.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the scope of the claims is intended to encompass equivalents to the quantities. In one embodiment, the term “about” means within 10% of the reported numerical value, preferably within 5% of the reported numerical value.

When a range of values is provided herein, it is intended to encompass the end-points of the range unless specifically stated otherwise. Numerical values used herein have the precision of the number of significant figures provided, following the standard protocol in chemistry for significant figures as outlined in ASTM E29-08 Section 6. For example, the number 40 encompasses a range from 35.0 to 44.9, whereas the number 40.0 encompasses a range from 39.50 to 40.49.

The term “plant growth” or “plant performance” as used herein refers to measures of plant development such as seed germination, seedling emergence, early radical or root and shoot growth, plant height, time to flowering, tillering (for grasses), days to maturity, vigor, biomass and yield.

The term “propagating material” as used herein means a seed or regenerable plant part. The term “regenerable plant part” means a part of the plant other than a seed from which a whole plant may be grown or regenerated when the plant part is placed in agricultural or horticultural growing media such as moistened soil, peat moss, sand, vermiculite, perlite, rock wool, fiberglass, coconut husk fiber, tree fern fiber, and the like, or even a completely liquid medium such as water. Regenerable plant parts commonly include rhizomes, tubers, bulbs and corms of such geophytic plant species as potato, sweet potato, yam, onion, dahlia, tulip, narcissus, etc. Regenerable plant parts include plant parts that are divided (e.g., cut) to preserve their ability to grow into a new plant. Therefore regenerable plant parts include viable divisions of rhizomes, tubers, bulbs and corms which retain meristematic tissue, such as an eye. Regenerable plant parts can also include other plant parts such as cut or separated stems and leaves from which some species of plants can be grown using horticultural or agricultural growing media. As referred to in the present disclosure and claims, unless otherwise indicated, the term “seed” includes both unsprouted seeds and seeds in which the testa (seed coat) still surrounds part of the emerging shoot and root. Foliage as defined in the present application includes all aerial plant organs, that is, the leaves, stems, flowers and fruit.

The term “rhizosphere” as used herein refers to the area of soil that immediately surrounds and is affected by the plant's roots.

The term “treating” as used herein means applying a biologically effective amount of a plant performance enhancing compound, or a composition containing the compound, to a seed or other plant propagating material, plant foliage or plant rhizosphere; related terms such as “treatment” are intended to be interpreted analogously.

As used herein the term “biologically effective amount” refers to that amount of a substance required to produce a desired effect on plant growth and yield. Effective amounts of the composition will depend on several factors, including treatment method, plant species, propagating material type and environmental conditions.

The term “agricultural composition” as used herein refers to one or more substances formulated for at least one agricultural application. Agricultural applications are any application that enhances plant performance, such as, for example, germination improvement, growth improvement, yield improvement, pest control, disease control and resistance to abiotic environmental stress.

The term “alkyl” means a linear or branched hydrocarbon group up to and including 20 carbon atoms. Common examples of such alkyl groups include methyl, ethyl, propyl, isopropyl, butyl, s-butyl, isobutyl, pentyl, neopentyl, hexyl, heptyl, isoheptyl, 2-ethylhexyl, cyclohexyl and octyl.

The term “aryl” is known in the art to mean a group defined as a monovalent radical formed conceptually by removal of a hydrogen atom from a hydrocarbon that is structurally composed entirely of one or more benzene rings. Common examples of such hydrocarbons include benzene, biphenyl, terphenyl, naphthalene, phenyl naphthalene, and naphthylbenzene. Aryl groups, as used herein, include aromatic carbocyclic groups having a single ring (e.g., phenyl), multiple rings (e.g., biphenyl), or multiple condensed rings in which at least one is aromatic, (e.g., 1,2,3,4-tetrahydronaphthyl, naphthyl, anthryl, or phenanthryl), which is optionally mono-, di-, or trisubstituted with, e.g., halogen, lower alkyl, lower alkoxy, lower alkylthio, trifluoromethyl, lower acyloxy, aryl, heteroaryl, and hydroxy. Also included are heteroaryl groups wherein heteroaryl is defined as 5-, 6-, or 7-membered aromatic ring systems having at least one hetero atom selected from the group consisting of nitrogen, oxygen and sulfur. Examples of heteroaryl groups are pyridyl, pyrimidinyl, pyrrolyl, pyrazolyl, pyrazinyl, pyridazinyl, oxazolyl, furanyl, quinolinyl, isoquinolinyl, thiazolyl, and thienyl, which can optionally be substituted with, e.g., halogen, lower alkyl, lower alkoxy, lower alkylthio, trifluoromethyl, lower acyloxy, aryl, heteroaryl, and hydroxy.

“Monoalkenyl” or “monoalkynyl” as used herein refers to the presence of a double or triple bond connecting two carbon atoms. Dialkenyl or polyalkenyl refers to the presence of two or more double bond connected carbon atoms, respectively. Polyalkynyl refers to two or more triple bond connected carbon atoms.

The term “unsubstituted arylene” refers to an arylene that has no substituents. The arylene can be, a heteroarylene, comprising one or two hetero atoms selected from nitrogen, oxygen, and sulfur; a naphthylene; a heteronaphthylene comprising one or two hetero atoms selected from nitrogen, oxygen, and sulfur; a divalent radical derived from two fused aromatic or hetero aromatic rings containing five or six atoms each and comprising one or two hetero atoms selected from nitrogen, oxygen, and sulfur; a biphenylene; and a heterobiphenylene comprising one or two hetero atoms selected from nitrogen, oxygen, and sulfur.

The term “substituted arylene” refers to an arylene with one or more substituents. The arylene may be a heteroarylene as described above Embodiments of the present invention include compounds that are synthetic lipochitooligosaccharide analogs (SLA), compositions containing the compounds, and the use of the compounds to enhance plant performance. The compounds are of the general structure (I):

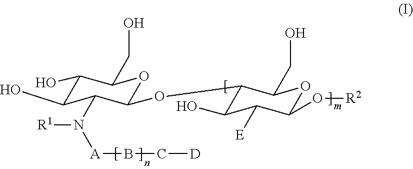

wherein the substituents are:
n is 1-20;
m is 0, 1, 2, 3 or 4; A is selected from —C(O)—, —C(S)—, C(O)O—, —C(O)S—, —C(S)S—;

B is selected from —C≡C—, —CR³═CR⁴—, and combinations thereof, wherein when present the carbon-carbon double bond configuration is selected from cis, trans, and a mixture of cis and trans;

C is a substituted or unsubstituted arylene wherein there are zero, one, two, three, or four substituents that are independently selected from a halogen, —CN, —C(O)OR⁵, —C(O)NR⁶R⁷, —CF₃, —OCF₃, —NO₂, —N₃, —OR⁵, —SR⁵, —NHR⁶, —NR⁶R⁷, and —C1-6 alkyl;

D is selected from H, CH₃, and a linear and branched, saturated and unsaturated, hydrocarbon-based chains containing from 2 to 20 carbon atoms;

E is selected from OH, NH₂, and NHC(O)CH₃;

R¹ is selected from H and C1-20 alkyl;

R² is selected from linear and branched, saturated and unsaturated, hydrocarbon-based chains containing from 1 to 20 carbon atoms, arylene, or substituted arylene;

R³ and R⁴ are independently selected from C1-6 alkyl, a halogen, and an aryl; and

R⁵, R⁶ and R⁷ are independently selected from H, —C1-6 alkyl, —C(O)C1-6 alkyl, —C(S)C1-6 alkyl, —C(O)OC1-6 alkyl, —C(O)NH₂, —C(S)NH₂, —C(NH)NH₂, —C(O)NHC1-6 alkyl, —C(S)NHC1-6 alkyl, and —C(NH)NHC1-6 alkyl.

In various embodiments, the substituted arylene of R² may be substituted with one or more substituents independently selected from: halogen, CN, C(O)OR⁵, C(O)NR⁶R⁷, CF₃, NO₂, C1-6 alkyl, mannose, glycerol, —C(O)C1-6 alkyl, —C(S)C1-6 alkyl, —C(O)OC1-6 alkyl, —C(O)NH₂, —C(S)NH₂, —C(NH)NH₂, —C(O)NHC1-6 alkyl, —C(S)NHC1-6 alkyl, and —C(NH)NHC1-6 alkyl.

In compounds of structure (I) the carbon chain of a aryl containing aliphatic carboxylic acid is attached directly to an amine substituent of an oligosaccharide, at the terminal amine-sugar unit. There is no aromatic ring between the carbon chain and the amine. The carbon chain is unsaturated, containing at least one alkenyl or one alkynyl group. There is an aromatic ring structure at the end of the carbon chain, which may itself have substituents, and is not directly attached to the oligosaccharide.

The [B]ₙ constituent, where B is selected from —C≡C—, CR³═CR⁴—, and combinations thereof, is a carbon chain that may include one or more triple bonds and/or one or more double bonds. The triple and/or double bonds may occur at any positions in the carbon chain that are chemically feasible.

The following structures are examples of compounds of structure (I) with substituent B specified, wherein all other substituents are as given above:

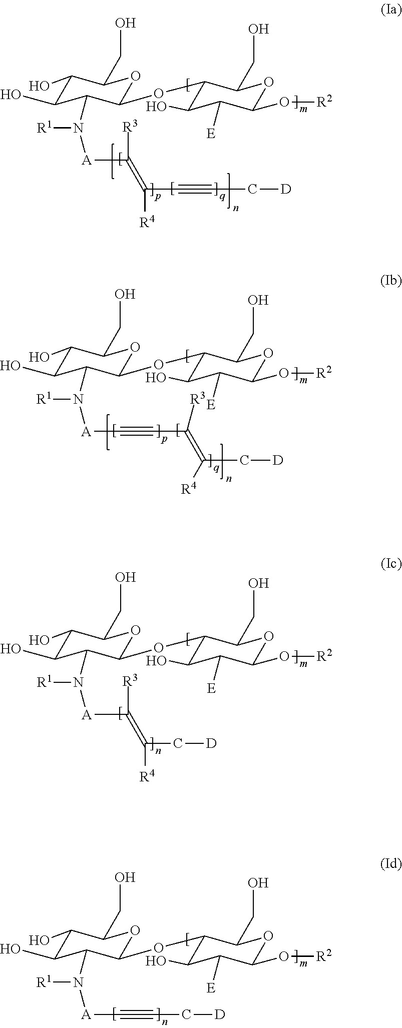

In various embodiments a compound of structure (I) has one or more of the following substituents:

n is 1 or 2;

m is 0 or 1;

A is —C(O)—;

B is selected from —C≡C— and —CH═CH—, wherein when present the carbon-carbon double bond configuration is selected from cis, trans, and a mixture of cis and trans;

C is an arylene substituted with one or more substituents selected from halogen, CN, CF₃, NO₂, and C1-6 alkyl;

D is selected from H, CH₃, and a linear and branched, saturated and unsaturated, hydrocarbon-based chains containing from 2 to 15 carbon atoms;

E is NHC(O)CH₃;

R¹ is selected from H and CH₃; and

R² is CH₃ or phenylene.

In one embodiment a compound of structure (I) has all of the following characteristics:

n is 1 or 2;

m is 0;

A is —C(O)—;

B is selected from —C≡C— and —CH═CH—, wherein when present the carbon-carbon double bond configuration is selected from cis, trans, and a mixture of cis and trans;

C is an arylene substituted with one or more substituents selected from halogen, CN, CF₃, NO₂, and C1-6 alkyl;

D is selected from H, CH₃, and a linear and branched, saturated and unsaturated, hydrocarbon-based chains containing from 2 to 15 carbon atoms;

R¹ is selected from H and CH₃; and

R² is CH₃ or phenylene.

Some specific examples of compounds of structure (I) include the following structures:

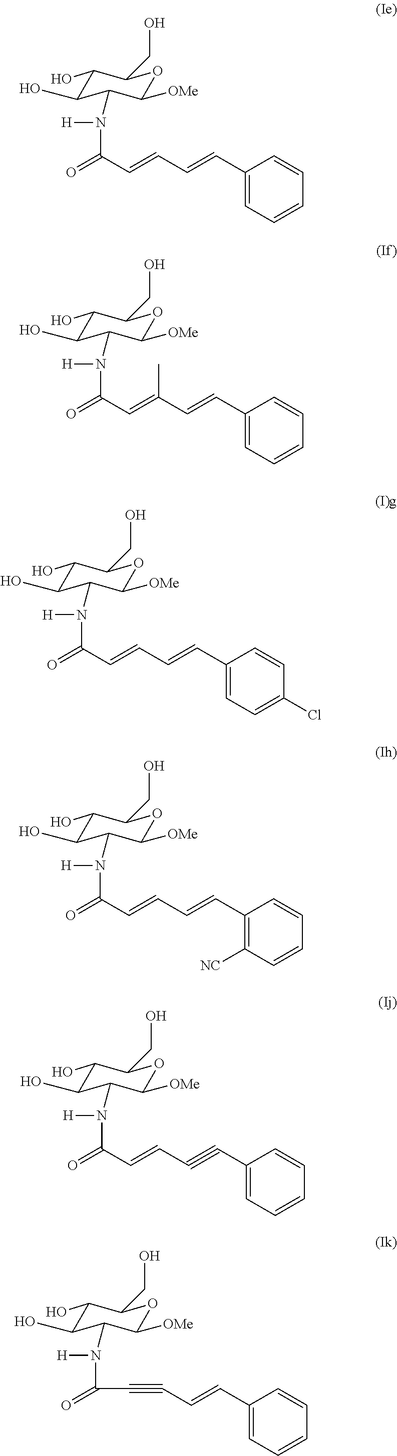 [SMILES: COC1OC(CO)C(O)C(O)C1NC(=O)/C=C/C=C/c1ccccc1 ; COC1OC(CO)C(O)C(O)C1NC(=O)/C=C(C)/C=C/c1ccccc1 ; COC1OC(CO)C(O)C(O)C1NC(=O)/C=C/C=C/c1ccc(Cl)cc1 ; COC1OC(CO)C(O)C(O)C1NC(=O)/C=C/C=C/c1ccccc1C#N ; COC1OC(CO)C(O)C(O)C1NC(=O)/C=C/C#Cc1ccccc1 ; COC1OC(CO)C(O)C(O)C1NC(=O)C#C/C=C/c1ccccc1]

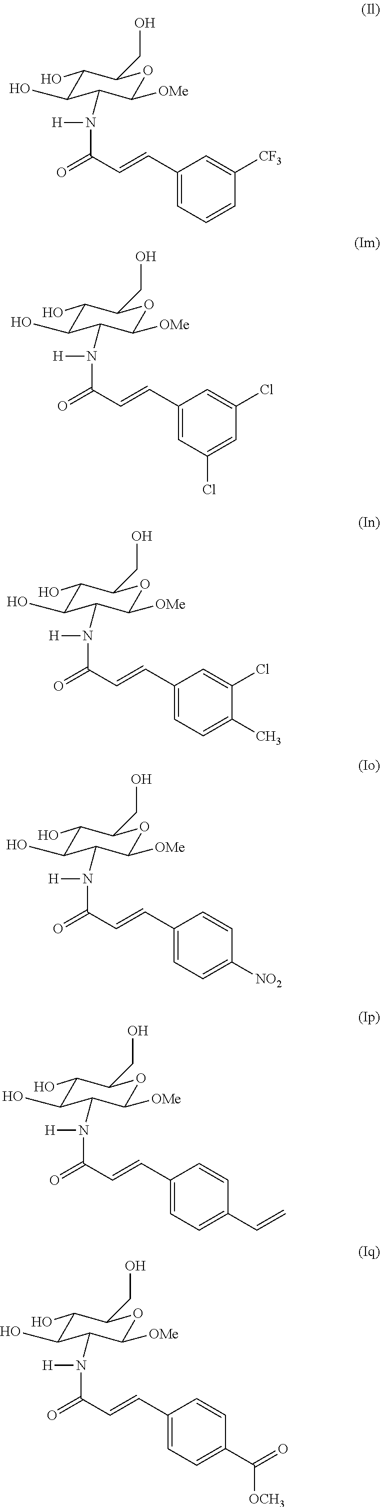 [SMILES: COC1OC(CO)C(O)C(O)C1NC(=O)/C=C/c1cccc(C(F)(F)F)c1 ; COC1OC(CO)C(O)C(O)C1NC(=O)/C=C/c1cc(Cl)cc(Cl)c1 ; COC1OC(CO)C(O)C(O)C1NC(=O)/C=C/c1ccc(C)c(Cl)c1 ; COC1OC(CO)C(O)C(O)C1NC(=O)/C=C/c1ccc([N+](=O)[O-])cc1 ; C=Cc1ccc(/C=C/C(=O)NC2C(OC)OC(CO)C(O)C2O)cc1 ; COC(=O)c1ccc(/C=C/C(=O)NC2C(OC)OC(CO)C(O)C2O)cc1]

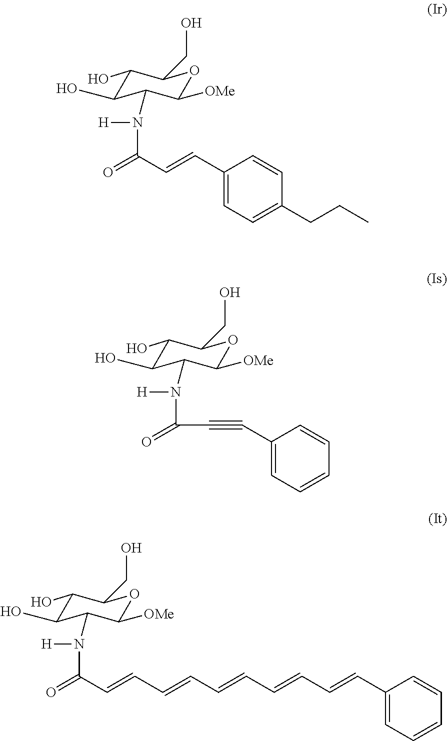 [SMILES: CCCc1ccc(/C=C/C(=O)NC2C(OC)OC(CO)C(O)C2O)cc1 ; COC1OC(CO)C(O)C(O)C1NC(=O)C#Cc1ccccc1 ; COC1OC(CO)C(O)C(O)C1NC(=O)/C=C/C=C/C=C/C=C/C=C/c1ccccc1]

Compounds of structure (I) can be synthesized by combining an aryl-containing aliphatic carboxylic acid of the desired structure, terminating with a desired substituted aromatic ring, and a desired oligosaccharide-amine, in the presence of 1-(3-dimethylaminopropyl)-3-ethylcarbodiimide (EDC) and 4-dimethylaminopyridine (DMAP). Typically the reaction is stirred overnight at room temperature, pumped dry, and the product is washed with water and dried. Synthesis of representative SLAs of structure (I) is described in examples herein.

Many of the desired aryl-containing aliphatic carboxylic acid acids are commercially available, for example from Alfa Aesar (Ward Hill, Mass.), 3B Scientific Corporation (Libertyville, Ill.), AK Scientific, Inc. (Union City, Calif.), and Aldrich Sigma (St. Louis, Mo.). If not commercially available, a desired acid can be synthesized starting with a commercially available alcohol, which can be obtained, for example, from the same sources given above. The alcohol is oxidized into aldehyde which is further reacted with a ylide to produce an alpha, beta-unsaturated ester. The resulting ester is hydrolyzed into the acid form, which is coupled with a oligosaccharide-amine as described above to form the desired SLA compound. These reactions are well known to one skilled in the art, and are described in, for example, Ireland and Norbeck (J. Org. Chem. (1985) 50:2198); Phillips et al. (Synlett (2008) 5:649-652); and Bressette and Glover (Synlett (2004) 4:738-740).

The oligosaccharide-amines can be synthesized as disclosed in U.S. Pat. No. 8,049,002, from commercial available D-glucosamine hydrochloride. For example, synthesis of oligoglucosamines is disclosed in U.S. Pat. No. 8,049,002.

In one embodiment, a compound of structure (I) is applied as a seed treatment formulation. Such formulations typically contain from about 10⁻⁵M to 10⁻¹²M of the compound of structure (I). In a preferred embodiment, formulations contain from about 10⁻⁶ M to 10⁻¹⁰ M of the compound. The locus of the propagating materials can be treated with the compound by many different methods. All that is needed is for a biologically effective amount of the compound of structure (I) to be applied on or sufficiently close to the propagating material so that it can be absorbed by the propagating material. The compound can be applied by such methods as drenching the growing medium including a propagating material with a solution or dispersion of the compound, mixing the compound with growing medium and planting a propagating material in the treated growing medium (e.g., nursery box treatments), or various methods of propagating material treatment whereby the compound of structure (I) is applied to a propagating material before it is planted in a growing medium.

In one embodiment a compound of structure (I) is present as an active ingredient in an agricultural composition. The agricultural composition is typically a formulation that includes an agriculturally suitable carrier comprising at least one of a liquid diluent, a solid diluent and a surfactant. A wide variety of formulations are suitable for the agricultural compositions; the most suitable types of formulations depend upon the method of application being used.

Formulations may include liquids such as solutions (including emulsifiable concentrates), suspensions, emulsions (including microemulsions and/or suspoemulsions) and the like which optionally can be thickened into gels. Useful formulations further include solids such as dusts, powders, granules, pellets, tablets, films, and the like which can be water-dispersible (“wettable”) or water-soluble. Active ingredient can be (micro)encapsulated and further formed into a suspension or solid formulation; alternatively the entire formulation of active ingredient can be encapsulated (or “overcoated”). Encapsulation can control or delay release of the active ingredient. Sprayable formulations can be extended in suitable media and used at spray volumes from about one to several hundred liters per hectare. High-strength compositions are primarily used as intermediates for further formulation.

The formulations will typically contain effective amounts of active ingredient, diluent and surfactant within the following approximate ranges that add up to 100 percent by weight.

| | Weight Percent | | |
|---|---|---|---|
| | Active | | |
| | Ingredient | Diluent | Surfactant |
| Water-Dispersible and | 5-90 | 0-94 | 1-15 |
| Water-soluble Granules, | | | |
| Tablets and Powders. | | | |
| Suspensions, Emulsions, | 5-50 | 40-95 | 0-15 |
| Solutions (including | | | |
| Emulsifiable Concentrates) | | | |
| Dusts | 1-25 | 70-99 | 0-5 |
| Granules and Pellets | 0.01-99 | 5-99.99 | 0-15 |
| High Strength Compositions | 90-99 | 0-10 | 0-2 |

Typical solid diluents are described in Watkins et al., Handbook of Insecticide Dust Diluents and Carriers, 2nd Ed., Dorland Books, Caldwell, N.J. Typical liquid diluents are described in Marsden, Solvents Guide, 2nd Ed., Interscience, New York, 1950. McCutcheon's Emulsifiers and Detergents and McCutcheon's Functional Materials (North America and International Editions, 2001), The Manufacturing Confection Publ. Co., Glen Rock, N.J., as well as Sisely and Wood, Encyclopedia of Surface Active Agents, Chemical Publ. Co., Inc., New York, 1964, list surfactants and recommended uses. All formulations can contain minor amounts of additives to reduce foam, caking, corrosion, microbiological growth and the like, or thickeners to increase viscosity.

Surfactants include, for example, ethoxylated alcohols, ethoxylated alkylphenols, ethoxylated sorbitan fatty acid esters, ethoxylated amines, ethoxylated fatty acids, esters and oils, dialkyl sulfosuccinates, alkyl sulfates, alkylaryl sulfonates, organosilicones, N,N-dialkyltaurates, glycol esters, phosphate esters, lignin sulfonates, naphthalene sulfonate formaldehyde condensates, polycarboxylates, and block polymers including polyoxyethylene/polyoxypropylene block copolymers.

Solid diluents include, for example, clays such as bentonite, montmorillonite, attapulgite and kaolin, starch, sugar, silica, talc, diatomaceous earth, urea, calcium carbonate, sodium carbonate and bicarbonate, and sodium sulfate. Liquid diluents include, for example, water, N,N-dimethylformamide, dimethyl sulfoxide, N-alkylpyrrolidone, ethylene glycol, polypropylene glycol, propylene carbonate, dibasic esters, paraffins, alkylbenzenes, alkylnaphthalenes, oils of olive, castor, linseed, tung, sesame, corn, peanut, cotton-seed, soybean, rape-seed and coconut, fatty acid esters, ketones such as cyclohexanone, 2-heptanone, isophorone and 4-hydroxy-4-methyl-2-pentanone, and alcohols such as methanol, cyclohexanol, decanol, benzyl and tetrahydrofurfuryl alcohol.

Solutions, including emulsifiable concentrates, can be prepared by simply mixing the ingredients. Dusts and powders can be prepared by blending and, usually, grinding as in a hammer mill or fluid-energy mill. Suspensions are usually prepared by wet-milling; see, for example, U.S. Pat. No. 3,060,084. Granules and pellets can be prepared by spraying the active material upon preformed granular carriers or by agglomeration techniques. See Browning, “Agglomeration”, Chemical Engineering, Dec. 4, 1967, pp. 147-48, Perry's Chemical Engineer's Handbook, 4th Ed., McGraw-Hill, New York, 1963, pp. 8-57 and following, and PCT Publication WO 91/13546. Pellets can be prepared as described in U.S. Pat. No. 4,172,714. Water-dispersible and water-soluble granules can be prepared as disclosed in U.S. Pat. No. 4,144,050, U.S. Pat. No. 3,920,442 and DE 3,246,493. Tablets can be prepared as disclosed in U.S. Pat. No. 5,180,587, U.S. Pat. No. 5,232,701 and U.S. Pat. No. 5,208,030. Films can be prepared as disclosed in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp. 81-96; and Hance et al., Weed Control Handbook, 8th Ed., Blackwell Scientific Publications, Oxford, 1989.

The compositions used for treating propagating materials, or plants grown therefrom, can also comprise (besides the compound of structure (I)) an effective amount of one or more other biologically active compounds or agents. Suitable additional compounds or agents include, but are not limited to, insecticides, fungicides, nematocides, bactericides, acaricides, entomopathogenic bacteria, viruses or fungi, growth regulators such as rooting stimulants, chemosterilants, repellents, attractants, pheromones, feeding stimulants and other signal compounds including apocarotenoids, flavonoids, jasmonates and strigolactones (Akiyama, et al., in Nature, 435:824-827 (2005); Harrison, in Ann. Rev. Microbiol., 59:19-42 (2005); Besserer, et al., in PLoS Biol., 4(7):e226 (2006); WO2009049747). These compounds can also be formulated into mixtures or multi-component formulations.

Examples of such biologically active compounds or agents with which compounds of Structure I disclosed herein can be mixed or formulated are: insecticides such as abamectin, acephate, acetamiprid, amidoflumet (S-1955), avermectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenproximate, fenvalerate, fipronil, flonicamid, flucythrinate, tau-fluvalinate, flufenerim (UR-50701), flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, noviflumuron (XDE-007), oxamyl, parathion, parathion-methyl, permethrin, phorate, phosalone, phosmet, phosphamidon, pirimicarb, profenofos, pymetrozine, pyridalyl, pyriproxyfen, rotenone, spinosad, spiromesifin (BSN 2060), sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron; fungicides such as acibenzolar, azoxystrobin, benomyl, blasticidin-S, Bordeaux mixture (tribasic copper sulfate), bromuconazole, carpropamid, captafol, captan, carbendazim, chloroneb, chlorothalonil, copper oxychloride, copper salts, cyflufenamid, cymoxanil, cyproconazole, cyprodinil, (S)-3,5-dichloro-N-(3-chloro-1-ethyl-1-methyl-2-oxopropyl)-4-methylbenzamide (RH 7281), diclocymet (S-2900), diclomezine, dicloran, difenoconazole, (S)-3,5-dihydro-5-methyl-2-(methylthio)-5-phenyl-3-(phenylamino)-4H-imidazol-4-one (RP 407213), dimethomorph, dimoxystrobin, diniconazole, diniconazole-M, dodine, edifenphos, epoxiconazole, famoxadone, fenamidone, fenarimol, fenbuconazole, fencaramid (SZX0722), fenpiclonil, fenpropidin, fenpropimorph, fentin acetate, fentin hydroxide, fluazinam, fludioxonil, flumetover (RPA 403397), flumorf/flumorlin (SYP-L190), fluoxastrobin (HEC 5725), fluquinconazole, flusilazole, flutolanil, flutriafol, folpet, fosetyl-aluminum, furalaxyl, furametapyr (S-82658), hexaconazole, ipconazole, iprobenfos, iprodione, isoprothiolane, kasugamycin, kresoxim-methyl, mancozeb, maneb, mefenoxam, mepronil, metalaxyl, metconazole, metominostrobin/fenominostrobin (SSF-126), metrafenone (AC 375839), myclobutanil, neo-asozin (ferric methanearsonate), nicobifen (BAS 510), orysastrobin, oxadixyl, penconazole, pencycuron, probenazole, prochloraz, propamocarb, propiconazole, proquinazid (DPX-KQ926), prothioconazole (JAU 6476), pyrifenox, pyraclostrobin, pyrimethanil, pyroquilon, quinoxyfen, spiroxamine, sulfur, tebuconazole, tetraconazole, thiabendazole, thifluzamide, thiophanate-methyl, thiram, tiadinil, triadimefon, triadimenol, tricyclazole, trifloxystrobin, triticonazole, validamycin and vinclozolin; nematocides such as aldicarb, oxamyl and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad; and biological agents including Bacillus thuringiensis (including ssp. aizawai and kurstaki), Bacillus thuringiensis delta-endotoxin, baculoviruses, and entomopathogenic bacteria, viruses and fungi. A general reference for these agricultural protectants is The Pesticide Manual, 12th Edition, C. D. S. Tomlin, Ed., British Crop Protection Council, Farnham, Surrey, U. K., 2000.

Preferred insecticides and acaricides for mixing or formulating with compounds of structure (I) include pyrethroids such as cypermethrin, cyhalothrin, cyfluthrin and beta-cyfluthrin, esfenvalerate, fenvalerate and tralomethrin; carbamates such as fenothicarb, methomyl, oxamyl and thiodicarb; neonicotinoids such as clothianidin, imidacloprid and thiacloprid; neuronal sodium channel blockers such as indoxacarb, insecticidal macrocyclic lactones such as spinosad, abamectin, avermectin and emamectin; γ-aminobutyric acid (GABA) antagonists such as endosulfan, ethiprole and fipronil; insecticidal ureas such as flufenoxuron and triflumuron; juvenile hormone mimics such as diofenolan and pyriproxyfen; pymetrozine; and amitraz. Preferred biological agents for mixing with the present compounds include Bacillus thuringiensis and Bacillus thuringiensis delta-endotoxin as well as naturally occurring and genetically modified viral insecticides including members of the family Baculoviridae as well as entomophagous fungi.

Preferred plant growth regulators for mixing or formulating with the compounds of structure (I) in compositions for treating stem cuttings are 1H-indole-3-acetic acid, 1H-indole-3-butanoic acid and 1-naphthaleneacetic acid and their agriculturally suitable salt, ester and amide derivatives, such as 1-napthaleneacetamide. Preferred fungicides for mixing with the structure (I) compounds include fungicides useful as seed treatments such as thiram, maneb, mancozeb and captan.

For growing-medium drenches, formulations can provide compound of structure (I), generally after dilution with water, in solution or as particles small enough to remain dispersed in the liquid. Water-dispersible or soluble powders, granules, tablets, emulsifiable concentrates, aqueous suspension concentrates and the like are formulations suitable for aqueous drenches of growing media. Drenches are most satisfactory for treating growing media that have relatively high porosity, such as light soils or artificial growing medium comprising porous materials such as peat moss, perlite, vermiculite and the like. The drench liquid comprising the compound of structure (I) can also be added to a liquid growing medium (i.e. hydroponics), which causes the compound to become part of the liquid growing medium. One skilled the art will appreciate that the amount of the compound of structure (I) needed in the drench liquid for efficacy (i.e. biologically effective amount) will vary with several factors including, but not limited to, plant species, propagating material type and environmental conditions. The concentration of the compound of structure (I) in the drench liquid is generally between about 10⁻⁵M to 10⁻¹²M of the composition, more typically between about 10⁻⁶ M to 10⁻¹⁰ M. One skilled in the art can easily determine the biologically effective concentration necessary for the desired level of efficacy.

For treating a growing medium, a compound of structure (I) can also be applied by mixing it as a dry powder or granule formulation with the growing medium. Because this method of application does not require first dispersing or dissolving in water, the dry powder or granule formulations need not be highly dispersible or soluble. While in a nursery box the entire body of growing medium may be treated, in an agricultural field only the soil in the vicinity of the propagating material is typically treated for environmental and cost reasons. To minimize application effort and expense, a formulation containing a compound of structure (I) is most efficiently applied concurrently with propagating material planting (e.g., seeding). For in-furrow application, the formulation (most conveniently a granule formulation) is applied directly behind the planter shoe. For T-band application, the formulation is applied in a band over the row behind the planter shoe and behind or usually in front of the press wheel. One skilled the art will appreciate that the amount of compound of structure (I) needed for efficacy (i.e. biologically effective amount) will vary with several factors including, but not limited to, plant species, propagating material type and environmental conditions. The concentration of compound of structure (I) in the growing medium locus is generally between about 10⁻⁵ M to 10⁻¹²M of the composition, more typically between about 10⁻⁶ M to 10⁻¹⁰ M. One skilled in the art can easily determine the biologically effective amount necessary for the desired level of efficacy.

A propagating material can be directly treated by soaking it in a solution or dispersion of a compound of structure (I). Although this application method is useful for propagating materials of all types, treatment of large seeds (e.g., having a mean diameter of at least 3 mm) is more effective than treatment of small seeds for providing efficacy. Treatment of propagating materials such as tubers, bulbs, corms, rhizomes and stem and leaf cuttings also can provide effective treatment of the developing plant in addition to the propagating material. The formulations useful for growing-medium drenches are generally also useful for soaking treatments. The soaking medium comprises a nonphytotoxic liquid, generally water-based although it may contain nonphytotoxic amounts of other solvents such as methanol, ethanol, isopropanol, ethylene glycol, propylene glycol, propylene carbonate, benzyl alcohol, dibasic esters, acetone, methyl acetate, ethyl acetate, cyclohexanone, dimethylsulfoxide and N-methylpyrrolidone, which may be useful for enhancing solubility of the compound of structure (I) and penetration into the propagating material. A surfactant can facilitate wetting of the propagating material and penetration of the compound. One skilled the art will appreciate that the amount of the compound needed in the soaking medium for efficacy (i.e. biologically effective amount) will vary with several factors including, but not limited to, plant species, propagating material type and environmental conditions. The concentration of the compound of structure (I) in the soaking liquid is generally between about 10⁻⁵M to 10⁻¹²M of the composition, more typically between about 10⁻⁶ M to 10⁻¹⁰ M. One skilled in the art can easily determine the biologically effective concentration necessary for the desired level of efficacy. The soaking time can vary from one minute to one day or even longer. Indeed, the propagating material can remain in the treatment liquid while it is germinating or sprouting (e.g., sprouting of rice seeds prior to direct seeding). As shoot and root emerge through the testa (seed coat), the shoot and root directly contact the solution comprising the compound of structure (I). For treatment of sprouting seeds of large-seeded crops such as rice, treatment times of about 8 to 48 hours, e.g., about 24 hours, is typical. Shorter times are most useful for treating small seeds.

A propagating material can also be coated with a composition comprising a biologically effective amount of a compound of structure (I). The coatings of the invention are capable of effecting a slow release of the compound by diffusion into the propagating material and surrounding medium. Coatings include dry dusts or powders adhering to the propagating material by action of a sticking agent such as methylcellulose or gum arabic. Coatings can also be prepared from suspension concentrates, water-dispersible powders or emulsions that are suspended in water, sprayed on the propagating material in a tumbling device and then dried. Compounds of structure (I) that are dissolved in the solvent can be sprayed on the tumbling propagating material and the solvent then evaporated. Such compositions preferably include ingredients promoting adhesion of the coating to the propagating material. The compositions may also contain surfactants promoting wetting of the propagating material. Solvents used must not be phytotoxic to the propagating material; generally water is used, but other volatile solvents with low phytotoxicity such as methanol, ethanol, methyl acetate, ethyl acetate, acetone, etc. may be employed alone or in combination. Volatile solvents are those with a normal boiling point less than about 100° C. Drying must be conducted in a way not to injure the propagating material or induce premature germination or sprouting.

The thickness of coatings can vary from adhering dusts to thin films to pellet layers about 0.5 to 5 mm thick. Propagating material coatings of this invention can comprise more than one adhering layer, only one of which need comprise a compound of structure (I). Generally pellets are most satisfactory for small seeds, because their ability to provide a biologically effective amount of the compound is not limited by the surface area of the seed, and pelleting small seeds also facilitates seed transfer and planting operations. Because of their larger size and surface area, large seeds and bulbs, tubers, corms and rhizomes and their viable cuttings are generally not pelleted, but instead coated with powders or thin films.

Propagating materials contacted with compounds of structure (I) in accordance to this invention include seeds. Suitable seeds include seeds of wheat, durum wheat, barley, oat, rye, maize, sorghum, rice, wild rice, cotton, flax, sunflower, soybean, garden bean, lima bean, broad bean, garden pea, peanut, alfalfa, beet, garden lettuce, rapeseed, cole crop, turnip, leaf mustard, black mustard, tomato, potato, pepper, eggplant, tobacco, cucumber, muskmelon, watermelon, squash, carrot, zinnia, cosmos, chrysanthemum, sweet scabious, snapdragon, gerbera, babys-breath, statice, blazing star, lisianthus, yarrow, marigold, pansy, impatiens, petunia, geranium and coleus. Of note are seeds of cotton, maize, soybean and rice. Propagating materials contacted with compounds of structure (I) in accordance to this invention also include rhizomes, tubers, bulbs or corms, or viable divisions thereof. Suitable rhizomes, tubers, bulbs and corms, or viable divisions thereof include those of potato, sweet potato, yam, garden onion, tulip, gladiolus, lily, narcissus, dahlia, iris, crocus, anemone, hyacinth, grape-hyacinth, freesia, ornamental onion, wood-sorrel, squill, cyclamen, glory-of-the-snow, striped squill, calla lily, gloxinia and tuberous begonia. Of note are rhizomes, tubers, bulbs and corms, or viable division thereof of potato, sweet potato, garden onion, tulip, daffodil, crocus and hyacinth. Propagating materials contacted with compounds structure (I) in accordance to this invention also include stems and leaf cuttings.

In one embodiment, a propagating material is coated with a composition comprising the compound of structure (I) and a film former or adhesive agent. Compositions that contain a biologically effective amount of a compound of structure (I) and a film former or adhesive agent, can further contain an effective amount of at least one additional biologically active compound or agent. Examples include compositions comprising (in addition to the compound of structure (I) component and the film former or adhesive agent) an arthropodicides selected from the group consisting of pyrethroids, carbamates, neonicotinoids, neuronal sodium channel blockers, insecticidal macrocyclic lactones, γ-aminobutyric acid (GABA) antagonists, insecticidal ureas and juvenile hormone mimics. Also of note are compositions comprising (in addition to the compound of structure (I) component and the film former or adhesive agent) at least one additional biologically active compound or agent selected from the group consisting of abamectin, acephate, acetamiprid, amidoflumet (S-1955), avermectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenproximate, fenvalerate, fipronil, flonicamid, flucythrinate, tau-fluvalinate, flufenerim (UR-50701), flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, noviflumuron (XDE-007), oxamyl, parathion, parathion-methyl, permethrin, phorate, phosalone, phosmet, phosphamidon, pirimicarb, profenofos, pymetrozine, pyridalyl, pyriproxyfen, rotenone, spinosad, spiromesifin (BSN 2060), sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron, aldicarb, oxamyl, fenamiphos, amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben, tebufenpyrad; and biological agents such as Bacillus thuringiensis (including ssp. aizawai and kurstaki), Bacillus thuringiensis delta-endotoxin, baculoviruses, and entomopathogenic bacteria, viruses and fungi. Also of note are compositions comprising (in addition to the compound of structure (I) component and the film former or adhesive agent) at least one additional biologically active compound or agent selected from fungicides of the group consisting of acibenzolar, azoxystrobin, benomyl, blasticidin-S, Bordeaux mixture (tribasic copper sulfate), bromuconazole, carpropamid, captafol, captan, carbendazim, chloroneb, chlorothalonil, copper oxychloride, copper salts, cyflufenamid, cymoxanil, cyproconazole, cyprodinil, (S)-3,5-dichloro-N-(3-chloro-1-ethyl-1-methyl-2-oxopropyl)-4-methylbenzamide (RH 7281), diclocymet (S-2900), diclomezine, dicloran, difenoconazole, (S)-3,5-dihydro-5-methyl-2-(methylthio)-5-phenyl-3-(phenylamino)-4H-imidazol-4-one (RP 407213), dimethomorph, dimoxystrobin, diniconazole, diniconazole-M, dodine, edifenphos, epoxiconazole, famoxadone, fenamidone, fenarimol, fenbuconazole, fencaramid (SZX0722), fenpiclonil, fenpropidin, fenpropimorph, fentin acetate, fentin hydroxide, fluazinam, fludioxonil, flumetover (RPA 403397), flumorf/flumorlin (SYP-L190), fluoxastrobin (HEC 5725), fluquinconazole, flusilazole, flutolanil, flutriafol, folpet, fosetyl-aluminum, furalaxyl, furametapyr (S-82658), hexaconazole, ipconazole, iprobenfos, iprodione, isoprothiolane, kasugamycin, kresoxim-methyl, mancozeb, maneb, mefenoxam, mepronil, metalaxyl, metconazole, metominostrobin/fenominostrobin (SSF-126), metrafenone (AC 375839), myclobutanil, neo-asozin (ferric methanearsonate), nicobifen (BAS 510), orysastrobin, oxadixyl, penconazole, pencycuron, probenazole, prochloraz, propamocarb, propiconazole, proquinazid (DPX-KQ926), prothioconazole (JAU 6476), pyrifenox, pyraclostrobin, pyrimethanil, pyroquilon, quinoxyfen, spiroxamine, sulfur, tebuconazole, tetraconazole, thiabendazole, thifluzamide, thiophanate-methyl, thiram, tiadinil, triadimefon, triadimenol, tricyclazole, trifloxystrobin, triticonazole, validamycin and vinclozolin (especially compositions wherein the at least one additional biologically active compound or agent is selected from fungicides in the group consisting of thiram, maneb, mancozeb and captan).

In some embodiments, a propagating material coating comprises a compound of structure (I), a film former or sticking agent. The coating may further comprise formulation aids such as a dispersant, a surfactant, a carrier and optionally an antifoam and dye. One skilled the art will appreciate that the amount of structure (I) compound needed for efficacy (i.e. biologically effective amount) will vary with several factors including, but not limited to, plant species, propagating material type and environmental conditions. It is desired that the coating not inhibit germination or sprouting of the propagating material.

The film former or adhesive agent component of the propagating material coating preferably contains an adhesive polymer that may be natural or synthetic and is without phytotoxic effect on the propagating material to be coated. The film former or sticking agent can be selected from polyvinyl acetates, polyvinyl acetate copolymers, hydrolyzed polyvinyl acetates, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxymethylcelluloses, hydroxypropylcellulose, hydroxymethylpropylcelluloses, polyvinyl-pyrrolidones, alginates, dextrins, malto-dextrins, polysaccharides, fats, oils, proteins, karaya gum, jaguar gum, tragacanth gum, polysaccharide gums, mucilage, gum arabics, shellacs, vinylidene chloride polymers and copolymers, soybean-based protein polymers and copolymers, lignosulfonates, acrylic copolymers, starches, polyvinylacrylates, zeins, gelatin, carboxymethylcellulose, chitosan, polyethylene oxide, acrylimide polymers and copolymers, polyhydroxyethyl acrylate, methylacrylimide monomers, alginate, ethylcellulose, polychloroprene and syrups or mixtures thereof. Preferred film formers and adhesive agents include polymers and copolymers of vinyl acetate, polyvinylpyrrolidone-vinyl acetate copolymer and water-soluble waxes. Particularly preferred are polyvinylpyrrolidone-vinyl acetate copolymers and water-soluble waxes. The above-identified polymers include those known in the art and for example some are identified as Agrimer® VA 6 and Licowax® KST. The amount of film former or sticking agent in the formulation is generally in the range of about 0.001 to 100% of the weight of the propagating material. For large seeds the amount of film former or sticking agent is typically in the range of about 0.05 to 5% of the seed weight; for small seeds the amount is typically in the range of about 1 to 100%, but can be greater than 100% of seed weight in pelleting. For other propagating materials the amount of film former or sticking agent is typically in the range of 0.001 to 2% of the propagating material weight.

Materials known as formulation aids may also be used in propagating material treatment coatings of the invention and are well known to those skilled in the art. Formulation aids assist in the production or process of propagating material treatment and include, but are not limited, to dispersants, surfactants, carriers, antifoams and dyes. Useful dispersants can include highly water-soluble anionic surfactants like Borresperse™ CA, Morwet® D425 and the like. Useful surfactants can include highly water-soluble nonionic surfactants like Pluronic® F108, Brij® 78 and the like. Useful carriers can include liquids like water and oils which are water-soluble such as alcohols. Useful carriers can also include fillers like woodflours, clays, activated carbon, diatomaceous earth, fine-grain inorganic solids, calcium carbonate and the like. Clays and inorganic solids which may be used include calcium bentonite, kaolin, china clay, talc, perlite, mica, vermiculite, silicas, quartz powder, montmorillonite and mixtures thereof. Antifoams can include water dispersible liquids comprising polyorganic siloxanes like Rhodorsil® 416. Dyes can include water dispersible liquid colorant compositions like Pro-Ized® Colorant Red. One skilled in the art will appreciate that this is a non-exhaustive list of formulation aids and that other recognized materials may be used depending on the propagating material to be coated and the compound of structure (I) used in the coating. Suitable examples of formulation aids include those listed herein and those listed in McCutcheon's 2001, Volume 2: Functional Materials, published by MC Publishing Company. The amount of formulation aids used may vary, but generally the weight of the components will be in the range of about 0.001 to 10000% of the propagating material weight, with the percentages above 100% being mainly used for pelleting small seed. For nonpelleted seed generally the amount of formulating aids is about 0.01 to 45% of the seed weight and typically about 0.1 to 15% of the seed weight. For propagating materials other than seeds, the amount of formulation aids generally is about 0.001 to 10% of the propagating material weight.

Conventional methods of applying seed coatings can be used to carry out the coating of the invention. Dusts or powders may be applied by tumbling the propagating material with a formulation comprising a compound of structure (I) and a sticking agent to cause the dust or powder to adhere to the propagating material and not fall off during packaging or transportation. Dusts or powders can also be applied by adding the dust or powder directly to the tumbling bed of propagating materials, followed by spraying a carrier liquid onto the seed and drying. Dusts and powders comprising the compound can also be applied by treating (e.g., dipping) at least a portion of the propagating material with a solvent such as water, optionally comprising a sticking agent, and dipping the treated portion into a supply of the dry dust or powder. This method can be particularly useful for coating stem cuttings. Propagating materials can also be dipped into compositions comprising structure (I) formulations of wetted powders, solutions, suspoemulsions, emulfiable concentrates and emulsions in water, and then dried or directly planted in the growing medium. Propagating materials such as bulbs, tubers, corms and rhizomes typically need only a single coating layer to provide a biologically effective amount of a compound of structure (I).

Propagating materials can also be coated by spraying a suspension concentrate directly into a tumbling bed of propagating materials and then drying the propagating materials. Alternatively, other formulation types like wetted powders, solutions, suspoemulsions, emulsifiable concentrates and emulsions in water may be sprayed on the propagating materials. This process is particularly useful for applying film coatings to seeds. Various coating machines and processes are available to one skilled in the art. Suitable processes include those listed in P. Kosters et al., Seed Treatment: Progress and Prospects, 1994 BCPC Monograph No. 57 and the references listed therein. Three well-known techniques include the use of drum coaters, fluidized bed techniques and spouted beds. Propagating materials such as seeds may be presized prior to coating. After coating the propagating materials are dried and then optionally sized by transfer to a sizing machine. These machines are known in the art for example, as a typical machine used when sizing corn (maize) seed in the industry.

For coating seed, the seed and coating material are mixed in any variety of conventional seed coating apparatus. The rate of rolling and coating application depends upon the seed. For large oblong seeds such as those of cotton, a satisfactory seed coating apparatus comprises a rotating type pan with lifting vanes turned at sufficient rpm to maintain a rolling action of the seed, facilitating uniform coverage. For seed coating formulations applied as liquids, the seed coating must be applied over sufficient time to allow drying to minimize clumping of the seed. Using forced air or heated forced air can facilitate an increased rate of application. One skilled in the art will also recognize that this process can be a batch or continuous process. A continuous process allows the seeds to flow continuously throughout the product run. New seeds enter the pan in a steady stream to replace coated seeds exiting the pan.

The seed coating process of the present invention is not limited to thin film coating and may also include seed pelleting. The pelleting process typically increases the seed weight from 2 to 100 times and can be used to also improve the shape of the seed for use in mechanical seeders. Pelleting compositions generally contain a solid diluent, which is typically an insoluble particulate material, such as clay, ground limestone, powdered silica, etc., to provide bulk in addition to a binder such as an artificial polymer (e.g., polyvinyl alcohol, hydrolyzed polyvinyl acetates, polyvinyl methyl ether, polyvinyl methyl ether-maleic anhydride copolymer, and polyvinylpyrrolidinone) or natural polymer (e.g., alginates, karaya gum, jaguar gum, tragacanth gum, polysaccharide gum, mucilage). After sufficient layers have been built up, the coat is dried and the pellets graded. A method for producing pellets is described in Agrow, The Seed Treatment Market, Chapter 3, PJB Publications Ltd., 1994.

Seed varieties and seeds with specific transgenic traits can be tested determine which seed treatment options and application rates may complement such varieties and transgenic traits in order to enhance yield. Further, the good root establishment and early emergence that results from the proper use of the compound of structure (I) seed treatment may result in more efficient nitrogen use, a better ability to withstand drought and an overall increase in yield potential of a variety or varieties containing a certain trait when combined with a seed treatment.

In another embodiment of the invention, the composition is applied within a foliar formulation. Such formulations will generally include at least one additional component selected from the group consisting of surfactants, solid diluents and liquid diluents, which serve as a carrier. The formulation ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspoemulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake. Effective foliar formulations will typically contain from about 10⁻⁶M to 10⁻¹²M of the composition. In a preferred embodiment, formulations contain from about 10⁻⁶ M to 10⁻¹⁰ M of the compound of structure (I).

In another embodiment of the invention, the composition is applied to soil either prior to or following planting of plant propagating materials. Compositions can be applied as a soil drench of a liquid formulation, a granular formulation to the soil, a nursery box treatment or a dip of transplants. Of note is a composition of the present invention in the form of a soil drench liquid formulation. Of further note is this method wherein the environment is soil and the composition is applied to the soil as a soil drench formulation. Other methods of contact include application of a compound or a composition of the invention by direct and residual sprays, aerial sprays, gels, seed coatings, microencapsulations, systemic uptake, baits, ear tags, boluses, foggers, fumigants, aerosols, dusts and many others. One embodiment of a method of contact is a dimensionally stable fertilizer granule, stick or tablet comprising a compound or composition of the invention. Effective soil formulations will typically contain from about 10⁻⁵M to 10⁻¹²M of the composition. In a preferred embodiment, formulations contain from about 10⁻⁶ M to 10⁻¹⁰ M of the compound of structure (I).

The method of this invention is applicable to virtually all plant species. Seeds that can be treated include, for example, wheat (Triticum aestivum L.), durum wheat (Triticum durum Desf.), barley (Hordeum vulgare L.), oat (Avena sativa L.), rye (Secale cereale L.), maize (Zea mays L.), sorghum (Sorghum vulgare Pers.), rice (Oryza sativa L.), wild rice (Zizania aquatica L.), millet (Eleusine coracana, Panicum miliaceum), cotton (Gossypium barbadense L. and G. hirsutum L.), flax (Linum usitatissimum L.), sunflower (Helianthus annuus L.), soybean (Glycine max Merr.), garden bean (Phaseolus vulgaris L.), lima bean (Phaseolus limensis Macf.), broad bean (Vicia faba L.), garden pea (Pisum sativum L.), peanut (Arachis hypogaea L.), alfalfa (Medicago sativa L.), beet (Beta vulgaris L.), garden lettuce (Lactuca sativa L.), rapeseed (Brassica rapa L. and B. napus L.), cole crops such as cabbage, cauliflower and broccoli (Brassica oleracea L.), turnip (Brassica rapa L.), leaf (oriental) mustard (Brassica juncea Coss.), black mustard (Brassica nigra Koch), tomato (Lycopersicon esculentum Mill.), potato (Solanum tuberosum L.), pepper (Capsicum frutescens L.), eggplant (Solanum melongena L.), tobacco (Nicotiana tabacum), cucumber (Cucumis sativus L.), muskmelon (Cucumis melo L.), watermelon (Citrullus vulgaris Schrad.), squash (Curcurbita pepo L., C. moschata Duchesne. and C. maxima Duchesne.), carrot (Daucus carota L.), zinnia (Zinnia elegans Jacq.), cosmos (e.g., Cosmos bipinnatus Cay.), chrysanthemum (Chrysanthemum spp.), sweet scabious (Scabiosa atropurpurea L.), snapdragon (Antirrhinum majus L.), gerbera (Gerbera jamesonii Bolus), babys-breath (Gypsophila paniculata L., G. repens L. and G. elegans Bieb.), statice (e.g., Limonium sinuatum Mill., L. sinense Kuntze.), blazing star (e.g., Liatris spicata Willd., L. pycnostachya Michx., L. scariosa Willd.), lisianthus (e.g., Eustoma grandiflorum (Raf.) Shinn), yarrow (e.g., Achillea filipendulina Lam., A. millefolium L.), marigold (e.g., Tagetes patula L., T. erecta L.), pansy (e.g., Viola cornuta L., V. tricolor L.), impatiens (e.g., Impatiens balsamina L.) petunia (Petunia spp.), geranium (Geranium spp.) and coleus (e.g., Solenostemon scutellarioides (L.) Codd). Not only seeds, but also rhizomes, tubers, bulbs or corms, including viable cuttings thereof, can be treated according to the invention from, for example, potato (Solanum tuberosum L.), sweet potato (Ipomoea batatas L.), yam (Dioscorea cayenensis Lam. and D. rotundata Poir.), garden onion (e.g., Allium cepa L.), tulip (Tulipa spp.), gladiolus (Gladiolus spp.), lily (Lilium spp.), narcissus (Narcissus spp.), dahlia (e.g., Dahlia pinnata Cay.), iris (Iris germanica L. and other species), crocus (Crocus spp.), anemone (Anemone spp.), hyacinth (Hyacinth spp.), grape-hyacinth (Muscari spp.), freesia (e.g., Freesia refracta Klatt., F. armstrongii W. Wats), ornamental onion (Allium spp.), wood-sorrel (Oxalis spp.), squill (Scilla peruviana L and other species), cyclamen (Cyclamen persicum Mill. and other species), glory-of-the-snow (Chionodoxa luciliae Boiss. and other species), striped squill (Puschkinia scilloides Adams), calla lily (Zantedeschia aethiopica Spreng., Z. elliottiana Engler and other species), gloxinia (Sinnigia speciosa Benth. & Hook.) and tuberous begonia (Begonia tuberhybrida Voss.). Stem cuttings can be treated according to this invention include those from such plants as sugarcane (Saccharum officinarum L.), carnation (Dianthus caryophyllus L.), florists chrysanthemum (Chrysanthemum mortifolium Ramat.), begonia (Begonia spp.), geranium (Geranium spp.), coleus (e.g., Solenostemon scutellarioides (L.) Codd) and poinsettia (Euphorbia pulcherrima Willd.). Leaf cuttings which can be treated according to this invention include those from begonia (Begonia spp.), african-violet (e.g., Saintpaulia ionantha Wendl.) and sedum (Sedum spp.). The above recited cereal, vegetable, ornamental (including flower) and fruit crops are illustrative, and should not be considered limiting in any way. For reasons of economic importance, preferred embodiments of this invention include wheat, rice, maize, barley, sorghum, oats, rye, millet, soybeans, peanuts, beans, rapeseed, canola, sunflower, sugar cane, potatoes, sweet potatoes, cassava, sugar beets, tomatoes, plantains and bananas, and alfalfa.

In any of the formulations and applications described above, more than one compound of structure (I) may be included. Any number and combination of compounds of structure (I) may be included in the agricultural compositions described.

Although the foregoing invention has been described in some detail by way of illustration and example for purposes of clarity of understanding, certain changes and modifications may be practiced within the scope of the appended claims.

### EXAMPLES

The present invention is further illustrated in the following Examples. It should be understood that these Examples, while demonstrating some preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations is as follows: “hr” means hour(s), “min” means minute(s), “L” means liter(s), “mL” means milliliter(s), “μL” means microliter(s), “mM” means millimolar, “M” means molar, “mmol” means millimoles, “g” means gram(s), “mg” means milligram(s), “ppm” means parts per million, “w/w” means weight/weight, “cSt” means centiStokes. “˜” means approximately, “cm” means centimeter, “m” means meter(s), “′” means inch(es), “DI” means deionized, “V/V” means volume to volume.

THF is tetrahydrofuran
DMF is dimethylformamide

### General Methods and Materials

### Chemicals

3-Methylcinnamic acid (98% purity), 4-methylcinnamic acid (99% purity), 5-phenyl-2,4-pentadienoic acid (98% purity), and 1-(3-dimethylaminopropyl)-3-ethylcarbodiimide hydrochloride (98% purity) were purchased from Alfa Aesar (Ward Hill, Mass.). THF (ACS grade) and DMF (ACS grade) were purchased from EMD (Gibbstocon, N.J.). 3-(Trifluoromethyl) cinnamic acid (97% purity) was purchased from Oakwood Products, Inc. (West Columbia, S.C.).

### Preparation of 4-methyl(2R,3S,4R,5R,6R)-5-amino-2-(hydroxymethyl)-6-methoxytetrahydro-2H-pyran-3,4-diol

4-methyl(2R,3S,4R,5R,6R)-5-amino-2-(hydroxymethyl)-6-methoxytetrahydro-2H-pyran-3,4-diol was prepared in five steps starting from commercially available D-glucosamine hydrochloride. First, the amine group in D-glucosamine hydrochloride was protected by reacting with phthalic anhydride under basic conditions (sodium hydroxide/triethylamine) at room temperature. The resulting crude product was converted into 2-deoxy-1,3,4,6-tetra-O-acetyl-2-phthalimido-D-glucopyranose in the presence of acetic anhydride and N,N-dimethylaminopyridine. Methanol, SnCl₄, and DCM were then used to convert the O-acetyl into an O-methyl group. In the next step, all of the acetyl protection groups were removed in the presence of NaOMe/MeOH to obtain a trihydroxy methylglycoside. Finally, the trihydroxy methylglycoside was heated with ethylenediamine modified Merrifield resin in n-butanol at 110° C. to produce the desired product.

¹H NMR spectra were recorded at 500 MHz Bruker machine. Chemical shifts are given in ppm relative to deuterated dimethyl sulfoxide (DMSO).

### Seed Germination Assay

Materials were all sterilized before use. An aqueous solution of the test compound (25 mL, 10⁻⁷ M in DI-water) was prepared for a set of five repeat experiments. Five Petri dishes and 100 soybean seeds were used to test one compound. A piece of Whatman filter paper was used to cover the inner side of each Petri dish to allow uniform distribution of testing solution.

Twenty soybean seeds were placed on the filter paper area of one Petri dish. 5 mL of the test compound solution was carefully poured in the Petri dish. Control experiments were set up the same way with 20 soybean seeds and 5 mL of DI-water per dish without any compounds. The lid was placed on the Petri dish and was sealed with Para-film. Five dishes with repeat experiments were stacked. One stack of dishes was wrapped twice with aluminum foil to prevent the seeds receiving any light. The stacks were transferred to an incubator maintained at room temperature and the seeds were germinated in dark.

After 24 hr, the stacks were pulled out for measurement. The number of germinated seeds was counted and the percent of germination on each dish was calculated. Radicle emergence was used as the germination indicator. The dishes were placed unwrapped at room temperature for one day and the number of germinated seeds was counted to assure seeds were able to germinate and different germination yields were not caused by bad seed quality. Seeds showing above 90% of germination were considered normal. The standard deviation of five repeats was calculated. Test results with 10% or lower standard deviation was considered good. Compounds acting as plant performance enhancers should promote a significant increase in average percentage of germination compared to the control above the standard deviation.

Plant growth assay Seeds germinated for 24 hr using the seed germination assay above were left open to light for another 40 hr. The radical length was measured and the percentage of germinating seed with radical length of greater than 1.5 cm was determined.

### Example 1

### Synthesis of (2E,4E)-N-((2R,3R,4R,5S,6R)-4,5-dihydroxy-6-(hydroxymethyl)-2-methoxytetrahydro-2H-pyran-3-yl)-5-phenylpenta-2,4-dienamide

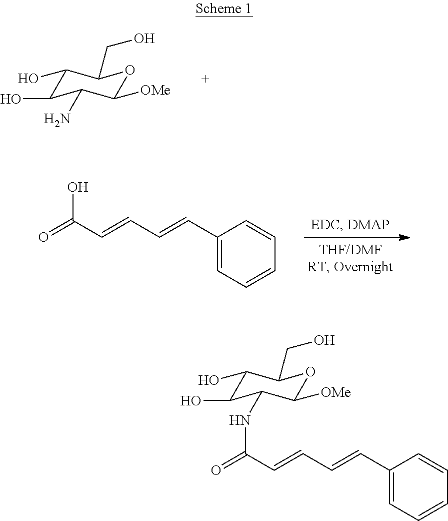 [SMILES: COC1OC(CO)C(O)C(O)C1N ; O=C(O)/C=C/C=C/c1ccccc1 ; COC1OC(CO)C(O)C(O)C1NC(=O)/C=C/C=C/c1ccccc1]

5-Phenyl-2,4-pentadienoic acid (0.1111 g, 0.63 mmol) was dissolved in a solvent mixture of DMF/THF (V/V=1/2, 3 mL total). To this solution, 4-dimethylaminopyridine (DMAP; 0.071 g, 0.58 mmol), 1-(3-dimethylaminopropyl)-3-ethylcarbodiimide (EDC) HCl (0.112 g, 0.58 mmol), and (2R,3S,4R,5R,6R)-5-amino-2-(hydroxymethyl)-6-methoxytetrahydro-2H-pyran-3,4-diol (0.1 g, 0.53 mmol) were added and the reaction was stirred overnight at room temperature. The reaction is diagrammed in Scheme 1.

The reaction mixture was pumped dry and washed with DI-H₂O 3 times (1 mL each). The purified product was dried under vacuum and analyzed with ¹H NMR and LC-MS. 50 mg of product was obtained in a yield of 27% based on the amount of the amine.

¹H NMR (500 MHz, DMSO-D6): δ7.95 (d, J=11.3 Hz, 1H), 7.55 (d, J=9.2 Hz, 2H), 7.37 (t, J=9.2 Hz, 2H), 7.30-7.27 (m, 1H), 7.18-6.91 (m, 3H), 6.14 (d, J=18.6 Hz, 1H), 5.03-4.96 (m, 2H), 4.62-4.59 (m, 1H), 4.26 (d, J=10.5 Hz, 1H), 3.73-3.69 (m, 1H), 3.56-3.46 (m, 3H), 3.32 (s, 3H), 3.12-3.10 (m, 2H). LC-MS (ESI): m/z 350 [M+1]⁺.

### Example 1A

### Testing of Soybean Seeds Treated with Example 1 Compound

(2E,4E)-N-((2R,3R,4R,5S,6R)-4,5-dihydroxy-6-(hydroxymethyl)-2-methoxytetrahydro-2H-pyran-3-yl)-5-phenylpenta-2,4-dienamide prepared in Example 1 was evaluated using the seed germination assay described in General Methods. Soybean seeds treated with this compound showed 75% germination in 24 hours. In the control experiment, soybean seeds showed 48% germination in 24 hours. The standard deviation of these experiments was below 10%.

The same compound was evaluated using the plant growth assay described in General Methods. Soybean seeds treated with this compound showed 69% of germinating seed with radical length longer than 1.5 cm after growing at room temperature for an additional 40 hr. The control soybean seeds had 45% with radical length longer than 1.5 cm after growing at room temperature for an additional 40 hr. The standard deviation of these experiments was below 10%.

### Example 2

### Synthesis of (E)-N-((2R,3R,4R,5S,6R)-4,5-dihydroxy-6-(hydroxymethyl)-2-methoxytetrahydro-2H-pyran-3-yl)-3-(p-tolyl)acrylamide

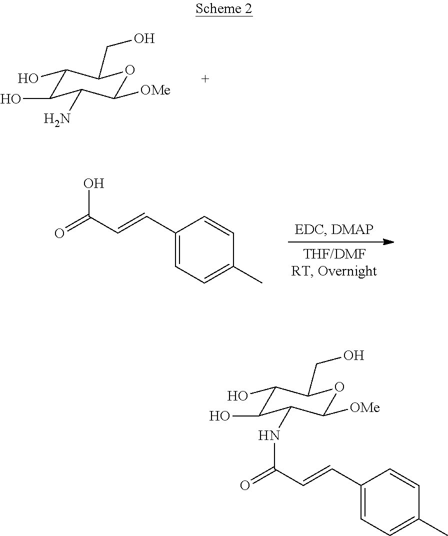 [SMILES: COC1OC(CO)C(O)C(O)C1N ; Cc1ccc(/C=C/C(=O)O)cc1 ; COC1OC(CO)C(O)C(O)C1NC(=O)/C=C/c1ccc(C)cc1]

4-Methylcinnamic Acid (0.103 g, 0.63 mmol) was dissolved in a solvent mixture of DMF/THF (V/V=1/2, 3 ml total). To this solution, DMAP (0.071 g, 0.58 mmol), EDC HCl (0.112 g, 0.58 mmol), and (2R,3S,4R,5R,6R)-5-amino-2-(hydroxymethyl)-6-methoxytetrahydro-2H-pyran-3,4-diol (0.1 g, 0.53 mmol) were added and the reaction was stirred overnight. The reaction is diagrammed in Scheme 2.

The reaction mixture was pumped dry and washed with DI-H₂O 3 times (1 mL/each). The purified product was dried under vacuum and analyzed with ¹H NMR and LC-MS. 97 mg of product was obtained in a yield of 55% based on the amount of the amine.

¹H NMR (500 MHz, DMSO-D6): δ 7.98 (d, J=11.4 Hz, 1H), 7.46 (d, J=10.6 Hz, 2H), 7.38 (d, J=19.7 Hz, 1H), 7.23 (d, J=10.6 Hz, 2H), 6.54 (d, J=19.7 Hz, 1H), 5.03-4.96 (m, 2H), 4.62-4.59 (m, 1H), 4.26 (d, J=10.5 Hz, 1H), 3.73-3.69 (m, 1H), 3.61-3.40 (m, 3H), 3.33 (s, 3H), 3.13-3.12 (m, 2H), 2.32 (s, 3H). LC-MS (ESI): m/z 338 [M+1]⁺.

### Example 2A

### Testing of Soybean Seeds Treated with Example 2 Compound

(E)-N-((2R,3R,4R,5S,6R)-4,5-dihydroxy-6-(hydroxymethyl)-2-methoxytetrahydro-2H-pyran-3-yl)-3-(p-tolyl)acrylamide prepared in Example 2 was evaluated using the seed germination assay described in General Methods. Soybean seeds treated with this compound showed 70% germination in 24 hours. In the control experiment, soybean seeds showed 48% germination in 24 hours. The standard deviation of these experiments was below 10%.

The same compound was evaluated using the plant growth assay described in General Methods. Soybean seeds treated with this compound showed 66% of germinating seed with radical length longer than 1.5 cm after growing at room temperature for an additional 40 hr. The control soybean seeds had 45% with radical length longer than 1.5 cm after growing at room temperature for an additional 40 hr. The standard deviation of these experiments was below 10%.

### Example 3

### Synthesis of (E)-N-((2R,3R,4R,5S,6R)-4,5-dihydroxy-6-(hydroxymethyl)-2-methoxytetrahydro-2H-pyran-3-yl)-3-(m-tolyl)acrylamide

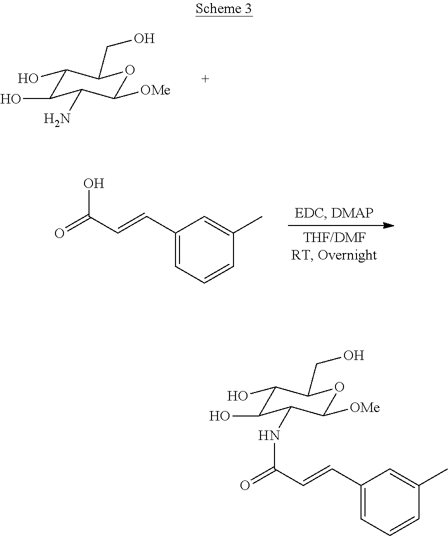 [SMILES: COC1OC(CO)C(O)C(O)C1N ; Cc1cccc(/C=C/C(=O)O)c1 ; COC1OC(CO)C(O)C(O)C1NC(=O)/C=C/c1cccc(C)c1]

3-Methylcinnamic Acid (0.103 g, 0.63 mmol) was dissolved in a solvent mixture of DMF/THF (V/V=1/2, 3 ml total). To this solution, DMAP (0.071 g, 0.58 mmol), EDC HCl (0.112 g, 0.58 mmol), and (2R,3S,4R,5R,6R)-5-amino-2-(hydroxymethyl)-6-methoxytetrahydro-2H-pyran-3,4-diol (0.1 g, 0.53 mmol) were added and the reaction was stirred overnight. The reaction is diagrammed in Scheme 3.

The reaction mixture was pumped dry and washed with DI-H₂O 3 times (1 mL/each). The purified product was dried under vacuum and analyzed with ¹H NMR and LC-MS. 60 mg of product was obtained in a yield of 34% based on the amount of the amine.

¹H NMR (500 MHz, DMSO-D6): δ 7.96 (d, J=9.1 Hz, 1H), 7.37-7.33 (m, 3H), 7.30 (t, J=7.6 Hz, 1H), 7.18 (d, J=7.6 Hz, 1H), 6.58 (d, J=15.8 Hz, 1H), 5.03-4.99 (m, 2H), 4.57-4.55 (m, 1H), 4.26 (d, J=8.5 Hz, 1H), 3.72-3.69 (m, 1H), 3.60-3.54 (m, 1H), 3.50-3.46 (m, 1H), 3.41-3.30 (m, 4H), 3.12-3.09 (m, 2H), 2.30 (s, 3H). LC-MS (ESI): m/z 338 [M+1]⁺.

### Example 3A

### Testing of Soybean Seeds Treated with Example 3 Compound

(E)-N-((2R,3R,4R,5S,6R)-4,5-dihydroxy-6-(hydroxymethyl)-2-methoxytetrahydro-2H-pyran-3-yl)-3-(m-tolyl)acrylamide prepared in Example 3 was evaluated using the seed germination assay described in General Methods. Soybean seeds treated with this compound showed 61% germination in 24 hours. In the control experiment, soybean seeds showed 48% germination in 24 hours. The standard deviation of these experiments was below 5%.

The same compound was evaluated using the plant growth assay described in General Methods. Soybean seeds treated with this compound showed 56% of germinating seed with radical length longer than 1.5 cm after growing at room temperature for an additional 40 hr. The control soybean seeds showed 45% with radical length longer than 1.5 cm after growing at room temperature for an additional 40 hr. The standard deviation of these experiments was below 5%.

### Example 4

### Synthesis of (E)-N-((2R,3R,4R,5S,6R)-4,5-dihydroxy-6-(hydroxymethyl)-2-methoxytetrahydro-2H-pyran-3-yl)-3-(3-(trifluoromethyl)phenyl)acrylamide

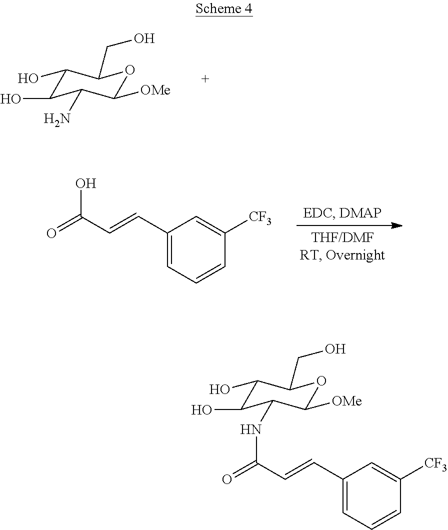 [SMILES: COC1OC(CO)C(O)C(O)C1N ; O=C(O)/C=C/c1cccc(C(F)(F)F)c1 ; COC1OC(CO)C(O)C(O)C1NC(=O)/C=C/c1cccc(C(F)(F)F)c1]

3-(Trifluoromethyl)cinnamic Acid (0.137 g, 0.63 mmol) was dissolved in a solvent mixture of DMF/THF (V/V=1/2, 3 ml total). To this solution, DMAP (0.071 g, 0.58 mmol), EDC HCl (0.112 g, 0.58 mmol), and (2R,3S,4R,5R,6R)-5-amino-2-(hydroxymethyl)-6-methoxytetrahydro-2H-pyran-3,4-diol (0.1 g, 0.53 mmol) were added and the reaction was stirred overnight. The reaction is diagrammed in Scheme 4.

The reaction mixture was pumped dry and washed with DI-H₂O 3 times (1 mL/each). The purified product was dried under vacuum and analyzed with ¹H NMR and LC-MS. 65 mg of product was obtained in a yield of 31% based on the amount of the amine.

¹H NMR (500 MHz, DMSO-D6): δ 8.04 (d, J=9.1 Hz, 1H), 7.90 (s, 1H), 7.87 (d, J=8.0 Hz, 1H), 7.71 (d, J=8.0 Hz, 1H), 7.64 (t, J=8.0 Hz, 1H), 7.48 (d, J=15.8 Hz, 1H), 6.73 (d, J=15.8 Hz, 1H), 5.0 (br s, 2H), 4.56 (br s, 1H), 4.25 (d, J=8.5 Hz, 1H), 3.72-3.70 (m, 1H), 3.61-3.56 (m, 1H), 3.49-3.47 (m, 1H), 3.46-3.29 (m, 4H), 3.13-3.11 (m, 2H). LC-MS (ESI): m/z 392 [M+1]⁺.

### Example 4A

### Testing of Soybean Seeds Treated with Example 4 Compound

(E)-N-((2R,3R,4R,5S,6R)-4,5-dihydroxy-6-(hydroxymethyl)-2-methoxytetrahydro-2H-pyran-3-yl)-3-(3-(trifluoromethyl)phenyl)acrylamide prepared in Example 4 was evaluated using the seed germination assay described in General Methods. Soybean seeds treated with this compound showed 75% germination in 24 hours. In the control experiment, soybean seeds showed 48% germination in 24 hours. The standard deviation of these experiments was below 10%.

The same compound was evaluated using the plant growth assay described in General Methods. Soybean seeds treated with this compound showed 69% of germinating seed with radical length longer than 1.5 cm after growing at room temperature for an additional 40 hr. The control soybean seeds had 45% with radical length longer than 1.5 cm after growing at room temperature for an additional 40 hr. The standard deviation of these experiments was below 10%.

### Example 5

### Synthesis of N-((2R,3R,4R,5S,6R)-4,5-dihydroxy-6-(hydroxymethyl)-2-methoxytetrahydro-2H-pyran-3-yl)-3-phenylpropiolamide

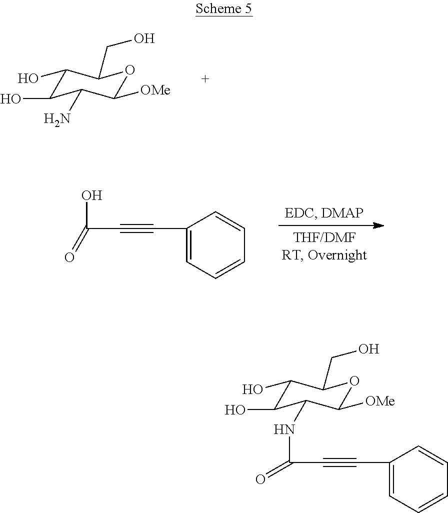 [SMILES: COC1OC(CO)C(O)C(O)C1N ; O=C(O)C#Cc1ccccc1 ; COC1OC(CO)C(O)C(O)C1NC(=O)C#Cc1ccccc1]

Phenylpropyoic acid (0.10 g, 0.68 mmol) was dissolved in a solvent mixture of DMF/THF (V/V=1/2, 3 mL total). To this solution, DMAP (0.12 g, 0.1.02 mmol), EDC HCl (0.2 g, 1.02 mmol), and (2R,3S,4R,5R,6R)-5-amino-2-(hydroxymethyl)-6-methoxytetrahydro-2H-pyran-3,4-diol (0.15 g, 0.75 mmol) were added and the reaction was stirred overnight at room temperature. The reaction is diagrammed in Scheme 5.

The reaction mixture was pumped dry and washed with DI-H₂O 3 times (1 mL/each). The purified product was dried under vacuum and analyzed with ¹H NMR and LC-MS. The reaction mixture was pumped dry and dissolved in DI-H₂O (2 ml) and centrifuged. 129 mg of product was obtained in a yield of 66% based on the amount of the phenylpropyoic acid.

¹H NMR (500 MHz, DMSO-D6): δ 8.63 (d, J=9.3 Hz, 1H), 7.59-7.57 (m, 2H), 7.53-7.45 (m, 3H), 5.09-5.05 (m, 2H), 4.59-4.57 (m, 1H), 4.22 (d, J=8.5 Hz, 1H), 3.71-3.68 (m, 1H), 3.56-3.46 (m, 3H), 3.35 (s, 3H), 3.09-3.06 (m, 2H). LC-MS (ESI): m/z 322 [M+1]⁺.

### Example 5A

### Testing of Soybean Seeds Treated with Example 5 Compound

N-((2R,3R,4R,5S,6R)-4,5-dihydroxy-6-(hydroxymethyl)-2-methoxytetrahydro-2H-pyran-3-yl)-3-phenylpropiolamide prepared in Example 5 was evaluated using the seed germination assay described in General Methods. Soybean seeds treated with this compound showed 86% germination in 24 hours. In the control experiment, soybean seeds showed 60% germination in 24 hours. The standard deviation of these experiments was below 10%.

The same compound was evaluated using the plant growth assay described in General Methods. Soybean seeds treated with this compound showed 95% of germinating seed with radical length longer than 1.5 cm after growing at room temperature for an additional 40 hrs. The control soybean seeds showed 66% with radical length longer than 1.5 cm after growing at room temperature for an additional 40 hrs. The standard deviation of these experiments was below 10%.

### Example 6

### Comparative

A comparative testing was done using the sugar structural unit, (2R,3S,4R,5R,6R)-5-amino-2-(hydroxymethyl)-6-methoxytetrahydro-2H-pyran-3,4-diol, to treat soybean seeds. In the seed germination assay performed as described in General Methods, the treated soybean seeds showed 53% germination in 24 hours. In the control experiment, soybean seeds showed 48% germination in 24 hours. The standard deviation of these experiments was below 10%.

The same compound was evaluated using the plant growth assay protocol as described in General Methods. Soybean seeds treated with this compound showed 45% of plants with radical length longer than 1.5 cm after growing at room temperature for an additional 40 hr. The control soybean seeds had 45% with radical length longer than 1.5 cm after growing at room temperature for an additional 40 hr. The standard deviation of these experiments was below 10%.

These results showed that there was insignificant efficacy of the sugar subunit by itself, which indicated the importance of both the sugar subunit and the acid subunit for the compound of Examples 1, 2, 3, 4, and 5 to be effective for enhanced seed germination and radical growth.

What is claimed is:

1. A compound having the structure (I) 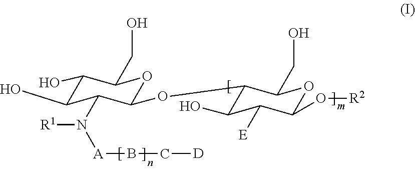
wherein the substituents are:
n is 1-20;
m is 0, 1, 2, 3 or 4;
A is selected from —C(O)—, —C(S)—, C(O)O—, —C(O)S—, —C(S)S—;
B is selected from —C≡C—, —CR³═CR⁴—, and combinations thereof, wherein when present the carbon-carbon double bond configuration is selected from cis, trans, and a mixture of cis and trans;
C is a substituted or unsubstituted arylene wherein there are one or more substituents that are independently selected from a halogen, —CN, —C(O)OR⁵, —C(O)NR⁶R⁷, —CF₃, —OCF₃, —NO₂, —N₃, —OR⁵, —SR⁵, —NHR⁶, —NR⁶R⁷, and —C1-6 alkyl;
D is selected from H, CH₃, and linear and branched, saturated and unsaturated, hydrocarbon-based chains containing from 2 to 20 carbon atoms;
E is selected from OH, NH₂, and NHC(O)CH3;
R¹ is selected from H and C1-20 alkyl;
R² is selected from linear and branched, saturated and unsaturated, hydrocarbon-based chains containing from 1 to 20 carbon atoms, arylene, and substituted arylene;
R³ and R⁴ are independently selected from halogens, C1-6 alkyl groups, and aryl groups; and
R⁵, R⁶ and R⁷ are independently selected from H, —C1-6 alkyl, —C(O)C1-6 alkyl, —C(S)C1-6 alkyl, —C(O)OC1-6 alkyl, —C(O)NH₂, —C(S)NH₂, —C(NH)NH₂, —C(O)NHC1-6 alkyl, —C(S)NHC1-6 alkyl, and —C(NH)NHC1-6 alkyl.

2. The compound of claim 1 having one or more of the following
n is 1 or 2;
m is 0 or 1;
A is —C(O)—;
B is selected from —C≡C— and —CH═CH—, wherein when present the carbon-carbon double bond configuration is selected from cis, trans, and a mixture of cis and trans;
C is an arylene substituted with one or more independently selected from halogen, CN, CF₃, NO₂, and C1-6 alkyl;
D is selected from H, CH₃, and linear and branched, saturated and unsaturated, hydrocarbon-based chains containing from 2 to 15 carbon atoms;
E is NHC(O)CH₃;
R¹ is selected from H and CH₃; and
R² is CH₃ or phenylene.

3. The compound of claim 2 wherein
n is 1 or 2;
m is 0;
A is —C(O)—;
B is selected from —C≡C— and —CH═CH—, wherein when present the carbon-carbon double bond configuration is selected from cis, trans, and a mixture of cis and trans;
C is an arylene substituted with one or more of independently selected from halogens, CN, CF₃, NO₂, and C1-6 alkyl;
D is selected from H, CH₃, and a linear and branched, saturated and unsaturated, hydrocarbon chains containing from 2 to 15 carbon atoms;
R¹ is selected from H and CH₃; and
R² is CH₃ or phenylene.

4. An agricultural composition comprising a compound having the structure (I) 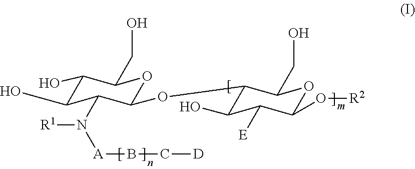
wherein:
n is 1-20;
m is 0, 1, 2, 3 or 4;
A is selected from —C(O)—, —C(S)—, C(O)O—, —C(O)S—, —C(S)S—;
B is selected from —C≡C—, —CR³═CR⁴—, and combinations thereof, wherein when present the carbon-carbon double bond configuration is selected from cis, trans, and a mixture of cis and trans;
C is a substituted or unsubstituted arylene wherein there are one or more substituents that are independently selected from halogens, —CN, —C(O)OR⁵, —C(O)NR⁶R⁷, —CF₃, —OCF₃, —NO₂, —N₃, —OR⁵, —SR⁵, —NHR⁶, —NR⁶R⁷, and —C1-6 alkyl;
D is selected from H, CH₃, and linear and branched, saturated and unsaturated, hydrocarbon-based chains containing from 2 to 20 carbon atoms;
E is selected from OH, NH₂, and NHC(O)CH₃;
R¹ is selected from H and C1-20 alkyl;
R² is selected from a linear and branched, saturated and unsaturated, hydrocarbon-based chains containing from 1 to 20 carbon atoms, arylene, and substituted arylene;
R³ and R⁴ are independently selected from C1-6 alkyl, a halogen, and an aryl; and
R⁵, R⁶ and R⁷ are independently selected from H, —C1-6 alkyl, —C(O)C1-6 alkyl, —C(S)C1-6 alkyl, —C(O)OC1-6 alkyl, —C(O)NH₂, —C(S)NH₂, —C(NH)NH₂, —C(O)NHC1-6 alkyl, —C(S)NHC1-6 alkyl, and —C(NH)NHC1-6 alkyl.

5. The agricultural composition of claim 4 wherein the compound having the structure (I) wherein
n is 1 or 2;
m is 0 or 1;
A is —C(O)—;
B is selected from —C≡C— and —CH═CH—, wherein when present the carbon-carbon double bond configuration is selected from cis, trans, and a mixture of cis and trans;
C is an arylene substituted with one or more selected from halogens, CN, CF₃, NO₂, and C1-6 alkyl;
D is selected from H, CH₃, and linear and branched, saturated and unsaturated, hydrocarbon-based chains containing from 2 to 15 carbon atoms;
E is NHC(O)CH₃;
R¹ is selected from H and CH₃; and
R² is CH₃ or phenylene.

6. The agricultural composition of claim 5 wherein
n is 1 or 2;
m is 0;
A is —C(O)—;
B is selected from —C≡C— and —CH═CH—, wherein when present the carbon-carbon double bond configuration is selected from cis, trans, and a mixture of cis and trans;
C is an arylene substituted with one or more selected from halogens, CN, CF₃, NO₂, and C1-6 alkyl;
D is selected from H, CH₃, and linear and branched, saturated and unsaturated, hydrocarbon-based chains containing from 2 to 15 carbon atoms;
R¹ is selected from H and CH₃; and
R² is CH₃ or phenylene.

7. The composition of claim 4 further comprising one or more selected from the group consisting of: insecticides, fungicides, nematocides, bactericides, acaricides, entomopathogenic bacteria, viruses, fungi, growth regulators and signal compounds.

8. The composition of claim 7 comprising a growth regulator selected from the group consisting of rooting stimulants, chemosterilants, repellents, attractants, pheromones, feeding stimulants and combinations thereof.

9. The composition of claim 7 comprising a signal compound selected from the group consisting of apocarotenoids, flavonoids, jasmonates, strigolactones, and combinations thereof.

10. A method of treating plant material comprising:
a) providing an agricultural composition of claim 4; and
b) contacting plant material with the composition.

11. The method of claim 8 wherein the contacting of (b) forms a seed coating.

12. The method of claim 10 wherein contacting of (b) is through application to soil either prior to or following planting plant propagating material.

13. The method of claim 10 wherein the agricultural composition of (a) further comprises one or more selected from the group consisting of: insecticides, fungicides, nematocides, bactericides, acaricides, entomopathogenic bacteria, viruses, fungi, growth regulators, and signal compounds.

14. The method of claim 13 wherein the growth regulator is selected from the group consisting of rooting stimulants, chemosterilants, repellents, attractants, pheromones, feeding stimulants, and combinations thereof.

15. The method of claim 13 wherein the signal compound is selected from the group consisting of apocarotenoids, flavonoids, jasmonates, strigolactones, and combinations thereof.
